(19) United States
(12) Patent Application Publication
(10) Pub. No.: US 2009/0162326 A1
(43) Pub. Date: Jun. 25, 2009

(54) METHOD FOR ISOLATING STEM CELLS AND STEM CELLS DERIVED FROM A PAD-LIKE TISSUE OF TEETH

(76) Inventors: Jurgen Siemonsmeier, Bonn (DE); Michael Thie, Bonn (DE); Ozer Degistirici, Bonn (DE); Werner Gotz, Bonn (DE)

Correspondence Address: JOYCE VON NATZMER;PEQUIGNOT + MYERS LLC, 200 Madison Avenue, Suite 1901, New York, NY, 10016, US

(21) Appl. No.: 11/997,245

(22) PCT Filed: Jul. 17, 2006
(86) PCT No.: PCT/EP2006/006994

§ 371 Date: Jul. 29, 2008

(30) Foreign Application Priority Data
Jul. 29, 2005 (EP) 05016549.7

(51) Int. Cl. A61K 45/00; C12N 5/00; C12N 5/08; A61P 43/00

(52) U.S. Cl. 424/93.7; 435/378; 435/366

(57) ABSTRACT

The invention relates to a method for isolating non-embryonic stem cells from a tissue that is located in immediate vicinity of immature, developing teeth or wisdom teeth. The invention further relates to non-embryonic stem cells derived from said tissue. The method according to the invention utilises a living soft tissue residing underneath the dental papilla 12 in immediate vicinity of the apical side of a developing tooth, which is clearly distinguished from other tooth tissue, such as dental papilla 12 or follicle. The pad-like tissue 16 can only be detected in a defined, specific developmental stage in an early phase of root formation. That is, identifying and separating the pad-like tissue 16 is only possible from the appearance of the bony alveolar fundus to the end of the formation of the root of the tooth.

### FIELD OF THE INVENTION

The invention concerns a method for isolating non-embryonic stem cells from a tissue that is located in immediate vicinity of immature, developing teeth or wisdom teeth. The invention further relates to non-embryonic stem cells derived from said tissue.

### BACKGROUND OF THE INVENTION

Stem cells are self-renewing cells that divide to give rise to a cell with an identical developmental potential and/or with a more restricted developmental potential, i.e. stem cells can divide asymmetrically to yield a stem cell and a more specialised cell which has lost some developmental potential. A stem cell has the ability to divide for indefinite periods, at least through many cycles, and often throughout the whole life of the organism. Given specific signals in the development of the organism, stem cells can differentiate into many different cell types that make up the organism. That is, stem cells have the potential to develop into mature cells that have characteristic shapes and specialised functions, such as bone cells, muscle cells, skin cells, or nerve cells. Unipotent stem cells produce a single type of differentiated cell, whereas pluripotent stem cells produce most cell types of the body.

Pluripotent stem cells derived from the inner cell mass of blastocysts (Embryonic Stem cells=ES cells) can give rise to cells that can form a wide range of cell types, such as the mesoderm, endoderm, and ectoderm of an embryo, and much attention has therefore been focused in the past on ES cells. However, ethical concerns have been raised about the use of embryonic stem cells. Thus, non-embryonic stem cells, i.e. somatic stem cells (Adult Stem cells=AS cells), are now of increasing importance for developing innovative therapeutic strategies.

An adult (somatic) stem cell (AS cell) is an undifferentiated cell that is situated in a differentiated tissue but can renew itself and become specialised to yield all of the specialised cell types of the tissue from which it originated, and possibly other specialised cells. For example, stem cells harvested from bone marrow have been used in therapy in cases of leukemia, lymphomas, and other life-threatening diseases. Recent studies have demonstrated that mesenchymal stem cells derived from bone marrow may be able to generate both skeletal muscle and neurons. Thus, there is evidence that AS cells derived from a specific type of tissue can generate mature, fully functional cells of another type, and that after exposure to a new environment, they may be able to populate other tissues and possibly differentiate into other cell types.

WO 01/60981 A1 discloses a method for producing tooth progenitor cells using cultured embryonic stem cells (ES cells). Production of the progenitor cells is here employed by inducing differentiation of the ES cells with oral epithelial cells. However, this approach uses embryos as a source for stem cells, so that beside ethical concerns also availability of the source material is limited.

WO 02/07676 A2 describes a method for regenerating human dentin and pulp using cultured adult stem cells (AS cells) derived from dental pulp. However, since non-embryonic stem cells from pulp are already in an advanced state of development they are not multipotent but merely differentiate into the specialised cell type of the tissue from which they were isolated. Moreover, obtaining the cells is very difficult because the tooth has to be cracked to make the pulp accessible.

WO 03/066840 A2 discloses a method for isolating non-embryonic stem cells derived from the dental follicle (dental sac) of tooth germs. The resulting stem cells are stated to be pluripotent and capable of differentiation into specialised cells of endodermal, ectodermal and mesodermal lineages. Although these stem cells may be a very promising parent material for several therapeutic approaches, isolation of the cells is difficult since only small amounts of follicle cells reside around a tooth. Moreover, this tissue is partially destroyed when the tooth is removed by usual surgical methods since parts of the dental follicle remain within the jaw attached to the tissue of the alveolar fundus surrounding the tooth.

### SUMMARY OF THE INVENTION

It is the object of the invention to provide a method for isolating non-embryonic stem cells, which can be easily accomplished and yields a considerable amount of cells, and non-embryonic stem cells that are at least multipotent and can be easily obtained in a considerable amount.

The object is met by a method for isolating non-embryonic stem cells comprising the steps of
identifying a pad-like tissue located underneath the dental papilla in immediate vicinity of the apical side of a surgically removed immature tooth or wisdom tooth which is in a development stage between the appearance of the bony alveolar fundus and the end of the formation of the root of said tooth or wisdom tooth,
separating said pad-like tissue from said dental papilla and/or said root, and
singularising cells by disintegrating said pad-like tissue.

The method according to the invention utilises a living soft tissue residing underneath the dental papilla in immediate vicinity of the apical side of a developing tooth, which is clearly distinguished from other tooth tissue, such as dental papilla or follicle (dental sac including operculum). In the stage of development between the appearance of the bony alveolar fundus and the end of the formation of the root, no dental follicle tissue is located at the apical side of the immature tooth but merely lateral follicle can be observed. That is, the follicular cells are either dissipated to form cementum, periodontal ligament and/or alveolar bone or migrated towards the crown. However, when a non-erupted, immature tooth has been surgically removed a pad-like tissue can be observed, which is attached to the dental papilla and/or the root at the apical side of the developing tooth. This valuable tissue is a white, pad-like soft tissue structure, which approximately has the size of a lentil or a bean, depending on the developmental stage. The pad-like tissue can only be detected in a defined, specific developmental stage in an early phase of root formation. That is, identifying and separating the pad-like tissue is only possible from the appearance of the bony alveolar fundus to the end of the formation of the root of the tooth.

In usual dental practice, merely the lateral and occlusal parts of the tissue surrounding the developing tooth, i.e. the remaining parts of the dental follicle, are admitted to histological analysis, whereas the apical soft tissue structure is discarded together with the immature tooth. That is, the potential of the soft tissue residing at the apical side of an immature, developing tooth was unknown by now and thus has been disregarded so far. However, once isolated using the method according to the invention the apical pad-like tissue shows its enormous potential as it surprisingly comprises at least multipotent stem cells which can differentiate as ectomesenchymal stem cells (mesenchymal cells derived from the ectodermal neural crest).

Identification and isolation of the pad-like tissue is easy and can be accomplished using appropriate standard surgical equipment. Since the pad-like tissue is a by-product of surgically removed non-erupted teeth it is easily available without limitation. Additionally, since the pad-like tissue has the size of a lentil or a bean, the method according to the invention yields a quantity of useful singularised cells that is sufficient for transplantation or other medical purposes. Moreover, the resulting stem cells are at least multipotent and can be used for producing functional cells and tissues of several types, even types that are different from tooth tissue. That is, the multipotent stem cells according to the invention can give rise to differentiated progeny and daughter cells that develop into all varieties of neural crest derivations.

In an advantageous embodiment of the invention, the pad-like tissue is separated from the dental papilla and/or the root by dissection, in particular by cutting along the macroscopically visible border between the dental papilla and the pad-like tissue. Thus, the pad-like tissue can be surgically harvested without using any sophisticated technical equipment. The border between the dental papilla and the pad-like tissue can be macroscopically detected and hence precise separation of the pad-like tissue can be accomplished. However, in some cases it may be difficult to exactly identify this border if the soft tissue attached to the surgically removed tooth has been affected, e.g. during transport. But even in those cases it is still possible at least to enrich the ectomesenchymal stem cells by surgically harvesting the soft tissue underneath the fictitious line between the roots of the tooth.

Subsequently, the pad-like tissue is disintegrated by enzymatic digestion, e.g. the cells are dissociated using collagenase and/or dispase in serum-free cell culture medium. The resulting singularised cells may be cultured and expanded in an appropriate medium, e.g. a standard cell culture medium containing fetal calf serum (FCS). The stem cells are highly proliferative and may be cultured as adhering cells over several cycles. The adhering cells are initially morphologically diverse, i.e. they comprise flat, disc-like cells, spindle-shaped cells, and spherical cells. In the end, fibroblast-like cells dominate the culture forming a confluent multilayer.

In a preferred embodiment of the invention, the tooth or wisdom tooth is a mammal tooth, preferably a human tooth. If the origin of the stem cells is human tooth tissue, the method according to the invention is the first step of promising cell therapies for humans, e.g. in respect of threatening disorders like cancer or degenerative diseases.

The singularised cells may be transfected with a nucleic acid, in particular DNA, preferably comprising at least one gene of interest and/or at least one marker sequence. Genetically engineering the resulting stem cells to express a gene of interest may be a beneficial approach for certain applications, for example, if a special therapy using transplanted stem cells has to be supported by a specific protein or peptide.

In a special embodiment of the invention, the singularised cells are stimulated to differentiate into bone-forming cells. In this case, osteogenic stimulation can be achieved by supplying the cells with 10⁻⁷ M dexamethasone, 50 μg/ml ascorbic acid 2-phosphate, and 10 mM β-glycerolphosphate. Bone-forming matrix can be detected, for example, by staining with alizarine red. As a result, the method according to the invention may be employed to achieve stem cells for regeneration or replacement of cranial bony tissue or parts of the skeletal system.

In another special embodiment of the invention, the singularised cells are stimulated to differentiate into neurons or nerve tissue. This can be achieved by expanding the singularised stem cells to high confluency and cultivating them in serum free medium with B27, bFGF and EGF.

The object is also met by a non-embryonic stem cell isolated from a pad-like tissue located underneath the dental papilla in immediate vicinity of the apical side of an immature tooth or wisdom tooth which is in a stage of development between the appearance of the bony alveolar fundus and the end of the formation of the root of said tooth or wisdom tooth. The present invention further comprises a non-embryonic stem cell, which is isolated by the method according to the invention. Such stem cells are at least multipotent and therefore able to produce functional cells and tissues of several developmental paths, even tissues that are different from tooth tissues. Since isolation of the pad-like tissue can be easily accomplished using appropriate standard equipment and the pad-like tissue has the size of a lentil or a bean, the non-embryonic stem cells according to the invention can be obtained in a considerable amount that is sufficient for transplantation or other medical purposes. The multipotent stem cells according to the invention can give rise to differentiated progeny and daughter cells that develop into all varieties of neural crest derivations. Hence, the stem cells according to the invention have the potential to generate lineages of neural crest-derivated cells and their differentiated cells. Since the ectomesenchymal stem cells according to the invention are derived from a tissue in an early development stage, e.g. in contrast to stem cells derived from pulp, they have an enormous potential as progenitor cells for many scientific and medical applications. Thus, the stem cells according to the invention differ from tooth-derived stem cells known from the prior art.

In a preferred embodiment of the invention, the stem cell according to the invention is a mammalian cell, preferably a human cell, and hence a promising source material for successful cell therapy for humans or other mammals.

In a particularly preferred embodiment of the invention, the non-embryonic stem cell is transfected with a nucleic acid, in particular DNA, preferably comprising at least one gene of interest and/or at least one marker sequence. That is, the present invention also relates to genetically engineered stem cells expressing at least one gene of interest.

The present invention also encompasses lineage-committed stem cells, either stem cell clones isolated by the method according to the invention or genetically engineered stem cells, which are at least multipotent and capable of producing cell lines that are committed to neural crest-derived lineages. In particular, the invention relates to isolation of at least multipotent somatic stem cells, preferably ectomesenchymal stem cells, and such isolated stem cells, respectively.

Any cell culture or cell structure comprising at least one non-embryonic stem cell according to the invention and pharmaceutical compositions comprising at least one non-embryonic stem cell according to the invention are subject to the invention as well. The cell cultures or cell structures which include at least one non-embryonic stem cell according to the invention can give rise to functional tissues or organs of several types. The pharmaceutical compositions according to the invention are based on the at least multipotent stem cells according to the invention and allow for treating various diseases or disorders resulting from cellular deficiencies. Possible therapies include repair or replacement of cells, tissues or organs by administration and/or transplantation of stem cells, cell structures or cell cultures according to the invention, or tissues or molecules derived therefrom. A cell therapy can be accomplished by administering to a patient, preferably a mammal, in particular a human being, an amount of the stem cells according to the invention, which is sufficient to cause a therapeutic effect. Besides usual auxiliary and/or carrier substances, the pharmaceutical compositions according to the invention may further comprise proliferation factors and/or lineage-commitment factors affecting the stem cells according to the invention.

The stem cells isolated by the method according to the invention or the stem cell according to the invention or the above-mentioned cell culture, cell structure, or pharmaceutical compositions each can be used for repairing or replacing damaged or destroyed tissue in vivo, for obtaining differentiated craniofacial cells or tissue including cells or tissue of the stromatognathic system, for obtaining differentiated neurons or nerve tissue, and/or for replacing or repairing ectomesenchymal tissue. Accordingly, as the stem cells are at least multipotent a wide range of applications can be covered based on the present invention.

The invention is described below in detail with reference to the drawings.

### BRIEF DESCRIPTION OF THE DRAWINGS

In the figures:

FIG. 1 represents a CT-Scan (CT=computed tomography) of a developing upper tooth, a) axial view, b) frontal view, c) sagittal view;

FIG. 2 represents a sagittal section through an unerupted developing human tooth;

FIG. 3 is a photograph of two surgically removed developing wisdom teeth (3^{rd} molar);

FIG. 4 shows a tissue section through the apical soft tissue of the wisdom tooth according to FIG. 3 showing histological details of the apical dental region, H.E. staining;

FIG. 5 represents a micrograph of isolated stem cells according to the invention;

FIG. 6 shows flow cytometric analyses of stem cells according to the invention, the cells being analysed for surface markers with CD 90, CD 73, CD 49e, CD 45, CD 31 and CD 13 specific antibodies, FITC=fluoresceinisothiocyanate-coupled antibody, PE=phycoerythrin-coupled antibody, APC=Allophycocyanin-coupled antibody;

FIG. 7 shows a periostin-specific staining of a tissue section through the apical soft tissue of a removed developing tooth;

FIG. 8 represents a micrograph of stem cells according to the invention after osteogenic stimulation, the sample being stained with alizarin red;

FIG. 9 is another photograph of a surgically removed phase I wisdom tooth (immature 3^{rd} molar);

FIG. 10 represents a micrograph of stem/progenitor cells according to the invention (EPCs) after Passage 20 (a), and a proliferation curve of EPCs (b);

FIG. 11 represents a flow cytometry analysis of stem/progenitor cells (EPCs) according to the invention after Passage 4 and Table 1 shows the results of the flow cytometry with EPCs compared to human bone marrow stem cells (hBMSC);

FIG. 12 represents a bar chart of a comparison of EPCs with fibroblasts after 21 days of osteogenic stimulation, Ca²⁺ production [μg/cm²] (a) and an agarose gel showing gene expression of EPCs after osteogenic induction, 0=day 0, 1=control, 2=osteogenically stimulated (b);

FIG. 13 represents a micrograph of toluidine blue staining of chondrogenic stimulated EPCs aggregate (a) and a gene expression analysis of chondrogenically stimulated EPCs, expression of GAPDH (internal reference), cartilage oligomeric protein (COMP), collagen type 11 (Col2A1) and aggrecan, 1=control EPCs and 2=treated EPCs (b);

FIG. 14 represents micrographs of stem/progenitor cells (EPCS) according to the invention after neurogenic induction (a), neurofilament positive cells (b), GFAP and MBP positive cells (c, d).

### VARIOUS AND PREFERRED EMBODIMENTS OF THE INVENTION

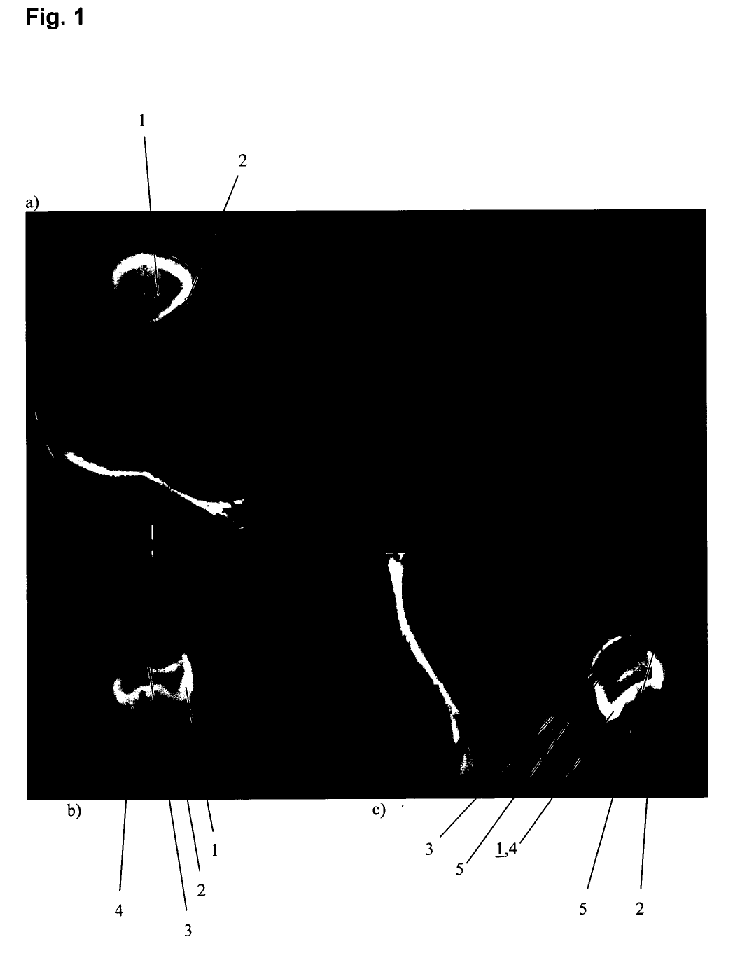

FIG. 1 represents a CT-Scan (CT=computed tomography) of a developing, unerupted upper tooth. The axial view a) shows the occlusal side of a developing tooth 1. That is, the coronary part including mucosa above the tooth 1 and operculum. The circumferential portion of the dental follicle 2 (lateral follicle) can also be observed in this representation. In frontal view b) and sagittal view c) of the tooth 1 a pad-like tissue 3 at the apical side (upper side in this representations) of the tooth 1 can be observed. The occlusal portion of the dental follicle 2 is located underneath the crown 4 of the tooth 1 diametrically opposed to the pad-like tissue 3. The pad-like tissue 3 according to the invention is situated above an imaginary line drawn between the roots 5 that are at the beginning of formation in this stage of tooth development.

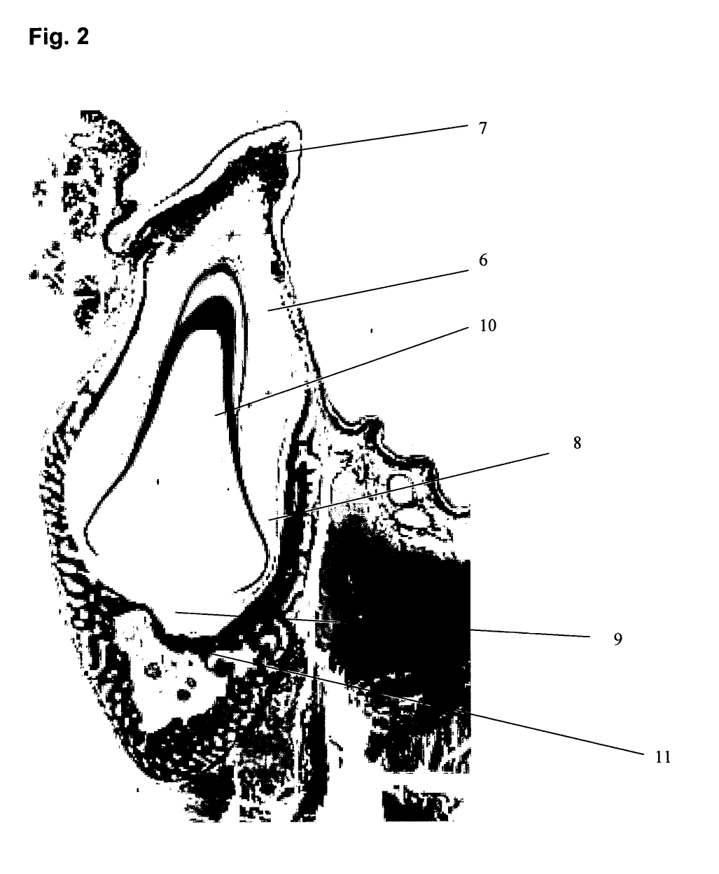

FIG. 2 represents a sagittal section through a developing unerupted human tooth 6. At the occlusal side of the tooth 6 the operculum 7 including mucosa can be observed, while the dental follicle 8 surrounds the lateral sides of the tooth 6. The pad-like tissue 9 is located at the apical side of the tooth 6 between the dental papilla 10 and the alveolar bone 11. Thus, at its apical side the pad-like tissue 9 contacts the occlusal surface of the alveolar bone 11.

According to the invention immature phase I or phase 11 teeth are used as source material. It is an advantage of this material that it is easily available in a significant number, particularly with children or adolescent persons. The developmental stage from the end of the formation of the crown 4 to the beginning of the formation of the root 5 is referred to as Phase I (see FIG. 1), while Phase II is the root forming stage. In order to avoid contamination with oral or subgingival bacteria the oral cavity has to be disinfected prior to removal of the tooth, for example by rinsing with “Betaisodona” or “Chlorhexamed” solution. After typical dissection and osteotomy the bony cover above the alveolar compartment is removed. Subsequently, the tooth is carefully removed and further treated with care.

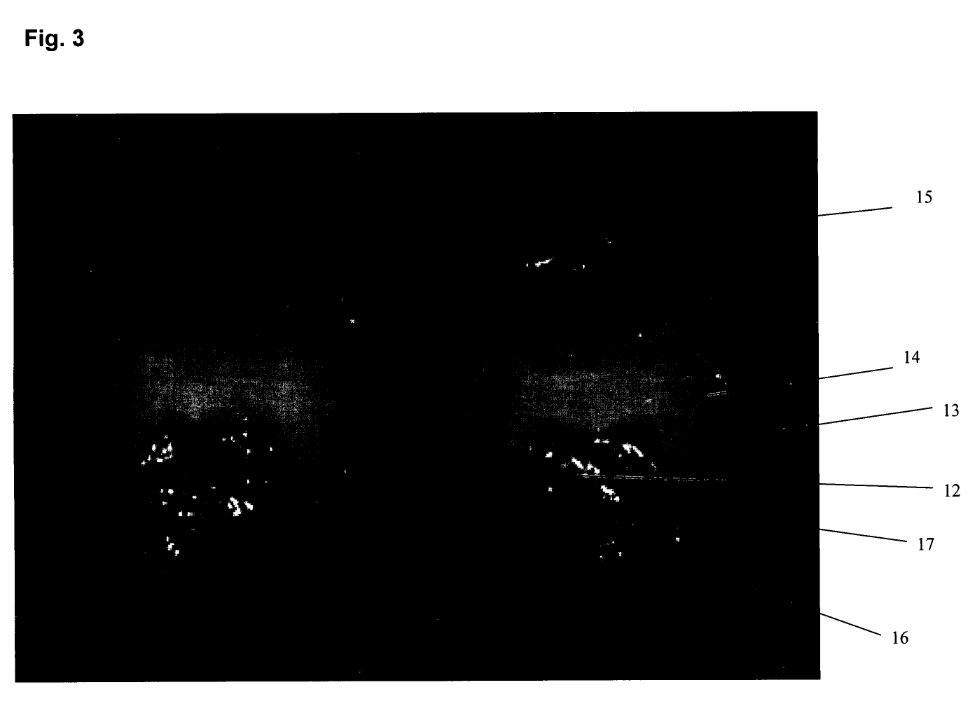

FIG. 3 is a photograph of surgically removed phase I wisdom teeth (3^{rd} molar). The tooth encompasses the dental papilla 12, the dental follicle 13, the enamel organ and its residual parts, respectively, as well as hard tissue of the crown 14 (enamel, dentine, and possibly cement). Under aseptic conditions, the coronary portion, i.e. mucosa above the tooth and operculum 15, is removed and discarded or used for histological analysis. The pad-like tissue 16 located at the apical side of the tooth underneath the dental papilla 12 is precisely separated from the dental papilla 12 and, if necessary, from the developing root 17, e.g. by cutting along the macroscopically visible border between the dental papilla 12 and the pad-like tissue 16 with a scalpel. The pad-like tissue 16 is a white, jelly-like tissue which approximately has the size of a lentil or a bean, depending on the stage of development of the developing tooth. This tissue comprises undifferentiated ectomesenchymal (neuroectodermal) stem cells.

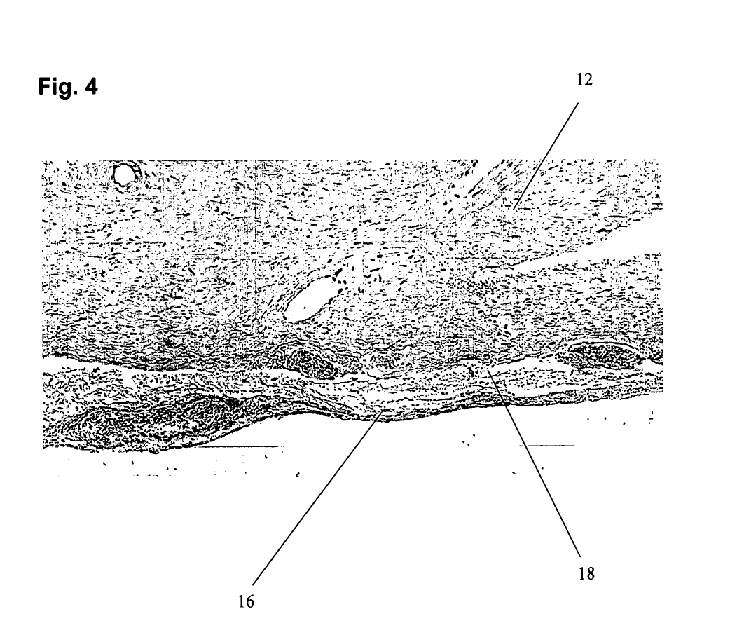

FIG. 4 shows a tissue section through the apical soft tissue of the wisdom tooth according to FIG. 3 showing histological details of the apical dental region by H.E. staining. This histological analysis shows that the pad-like tissue 16 is a connective tissue with a low level of collagen fibres. While no adipose tissue can be observed in the pad-like tissue 16, it shows capillary blood vessels and nerve fibres. Histologically, condensed connective tissue or loose, vascularized connective tissue can be observed in the border region 18 between the dental papilla 12 and the occlusal side of the pad-like tissue 16. This border region 18 approximately corresponds to the location of the epithelial diaphragm which is a process of Hertwig's epithelial borderline that turns from lateral to central and relates to root formation with multi-root teeth in later stages of development. In contrast to the dental papilla 12, which has a high cell density and includes numerous blood vessels and nerve fibres, the apical pad-like tissue 16 is characterised by a low cell density, a low matrix level and a lower level of blood vessels and nerve fibres. The cells of the pad-like tissue 16 are flat and spindle-shaped and aligned parallel to the surface of the tissue in the apical border zone. In contrast, the cells of the dental papilla 12 are star-shaped cells having spherical nuclei, which are randomly distributed in the tissue.

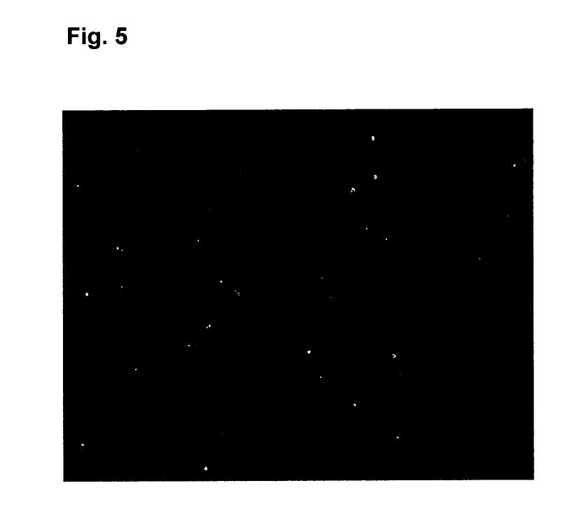

FIG. 5 represents a micrograph of isolated stem cells according to the invention. To this end, separated pad-like tissue was digested with collagenase/dispase in serum-free medium for 1 h at 37° C. After disintegrating the tissue, the singularised cells were placed in a T25 cell culture flask in a medium comprising DMEM low glucose (1 g/l), 10% fetal calf serum (FCS) and 1% penicillin/streptomcin/glutamine, and cultured at 37° C. in a humidified atmosphere of 5% CO₂. After 7 to 14 days of incubation, formation of single colonies of fibroblastoid cells occurred (FIG. 5). When the cells reached approximately 50 to 60% confluency they were passaged to a new culture flask (5,000 cells per cm²) and cultured further. The cells of the pad-like tissue according to the invention can be easily cultured as adhering cells and are highly proliferative. The adhering cells can be classified into morphologically different cell types, i.e. flat, disc-like cells, spindle-shaped cells and spherical cells. These cells show a heterogeneous appearance which changes in the course of time until, in the end, fibroblast-like cells dominate the culture forming a confluent multilayer. A cell population can be described by Vim⁺/nestin⁺/PanZyto⁻classification, which forms single nodule-like structures in a monolayer culture. Using ultrastructure analysis it can be demonstrated that these nodule-like structures encompass multilayered cells (fibroblasts/fibrocytes) which produce a loose extracellular matrix (collagen) and additionally further proteins (proteoglycans).

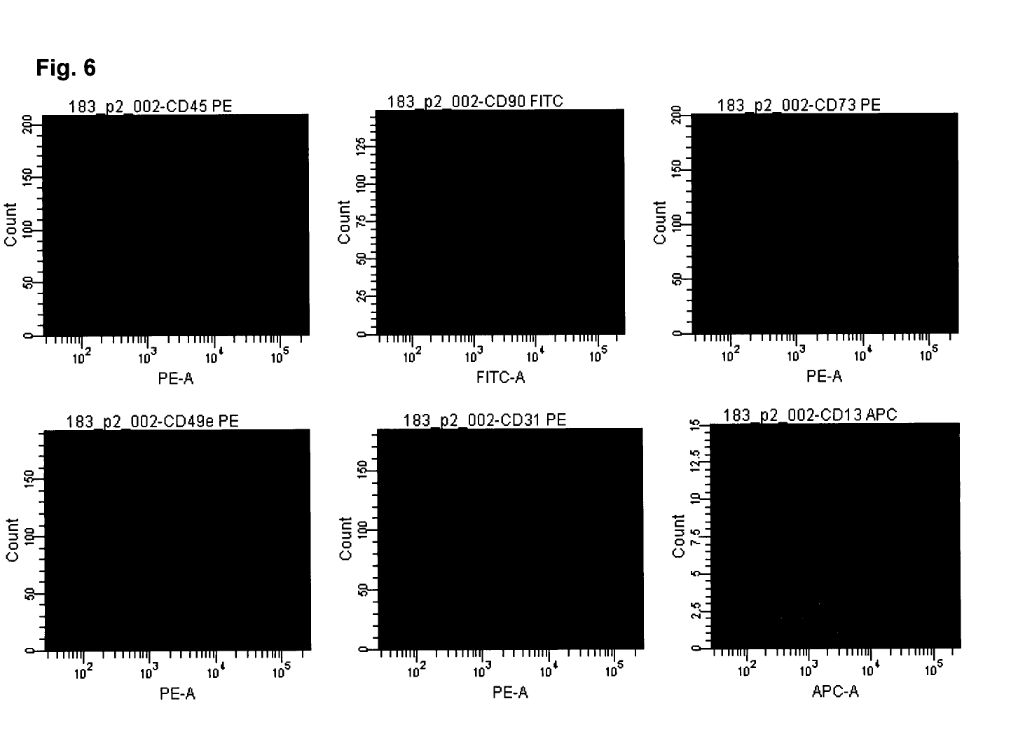

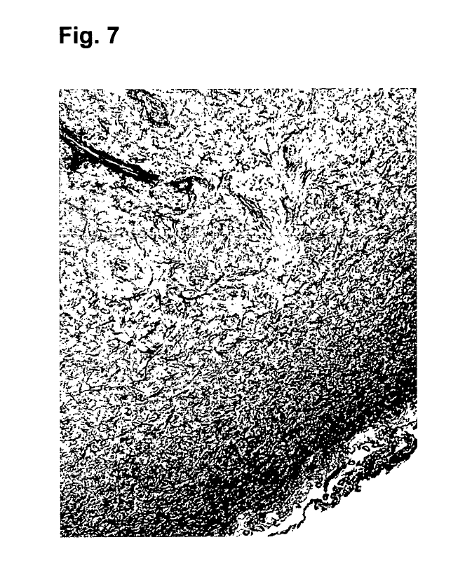

FIG. 6 shows flow cytometric analyses of stem cells according to the invention, wherein the cells are analysed for surface markers with CD 90, CD 73, CD 49e, CD 45, CD 31 and CD 13 specific antibodies. Approximately 60 surgically removed, non-pathological teeth were analysed histochemically using PAS and alcian blue staining and immunohistochemically using different antibodies. In these assays, marker for mesenchymal, endothelial and hematopoietic stem and progenitor cells, e.g. CD-90, CD-73, CD-49e, CD-45, CD-31, and CD-13 (FIG. 6), intermediate filaments (e.g. vimentin, keratins), as well as marker for differentiation of fibroblastic, osteoblastic and cementoblastic cells, e.g. BSP, ostoepontin, collagen, fibronectin, periostin, CAP (FIG. 7), could be detected.

Preliminary results indicate that the pad-like tissue contains more proteoglycans than dental pulp tissue and that discrimination from the neighbouring tissues may be possible using markers CD 57, p75, CAP as well as osteopontin and BSP.

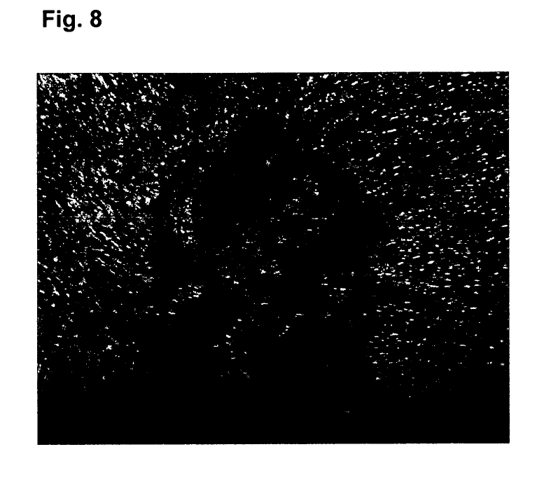

FIG. 8 represents a micrograph of stem cells according to the invention after osteogenic stimulation which can be achieved by supplying the cells with 10⁻⁷ M dexamethasone, 50 μg/ml ascorbic acid 2-phosphate and 10 mM β-glycerolphosphate. Bone-forming matrix can be detected by staining with alizarine red. The treated cells show significant calcium accumulation indicating the successful differentiation. This result demonstrates that the stem cells according to the invention can be stimulated for differentiation and that these cells are at least capable to give rise to osteoblasts.

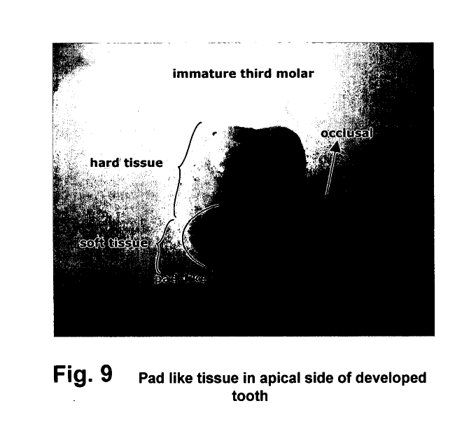

FIG. 9 is another photograph of a surgically removed phase I wisdom tooth (immature 3^{rd} molar) showing the pad-like soft tissue at the apical side of the removed tooth. The pad-like tissue is located at the apex of the tooth adjacent to the dental papilla. It is a white, jelly-like tissue which approximately has the size of a lentil or a bean, depending on the stage of development of the immature tooth.

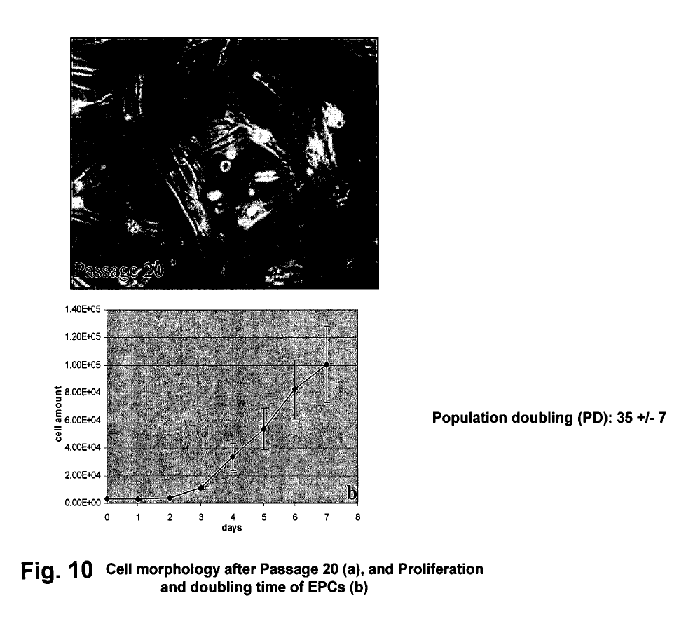

FIG. 10 represents a micrograph of stem/progenitor cells according to the invention (EPCs) showing the cell morphology after Passage 20 (a), and a proliferation curve showing the doubling time of EPCs, population doubling (PD)=35⁺/₋7 (b). Pad-like tissue from tooth of adolescents 11 to 18 years of age was used in this approach. Samples were processed within 2-4 hours. After collagenase/dispase digestion, suspended cells and remaining tissue were placed into culture medium (DMEM and 10% FCS). The primary cells grew as fibroblastic cells. After 15⁺/₋6 days, cell cultures reached 70-80% confluency (passage 0). Cells can be expanded up to passage 20 in culture (a). Ectomesenchymal progenitor cells (EPCs) (passage 3) showed growth curves characterized by an initial lag phase of 2 days, followed by an exponential log phase for 3-5 days. The mean cumulative time of population doublings (PD) of EPCs was about 35±7 hours (b).

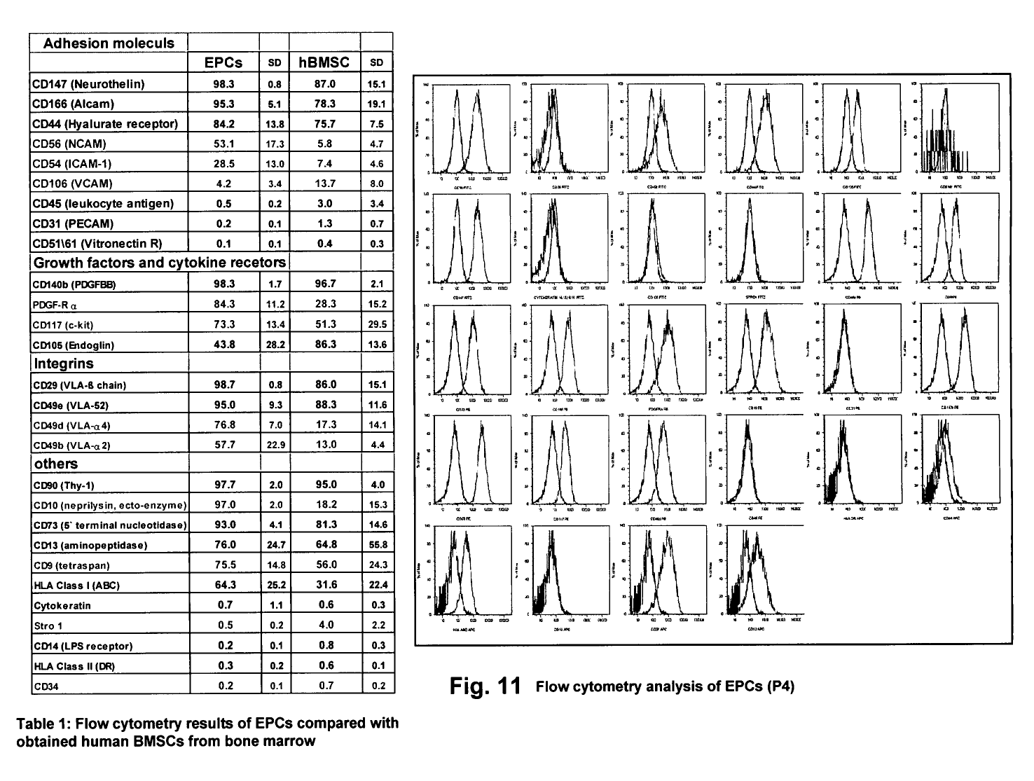

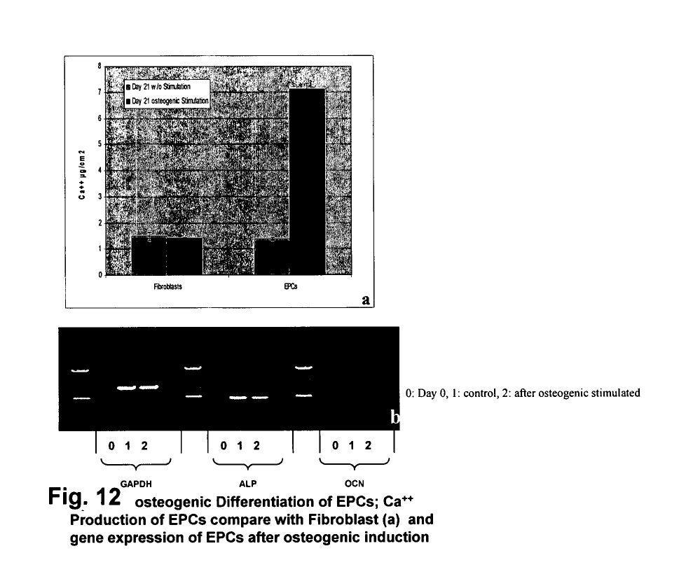

FIG. 11 represents a flow cytometry analysis of stem/progenitor cells (EPCs) according to the invention after Passage 4 and Table 1 shows the results of the flow cytometry with EPCs compared to human bone marrow stem cells (hBMSC). The CD marker profile of EPCs (passage 3-4) was determined and data were compared with commercially available human MSCs. EPCs showed high level of CD9, CD10, CD13, CD29, CD44, CD49b, CD49d, CD49e, CD56, CD73, CD90, CD105, CD117, CD140b, CD147, CD166, PDGFR a and class I HLA. Cells were moderately positive for CD54 and CD106. They did not express CD14, CD31, CD34, CD51/61, CD45, STRO 1, Cytokeratin (14/15/16/19), and class 11 HLA (FIG. 11). Compared to MSCs (and DSPCs, SHEDS), EPCs are unique in phenotype: They express CD56, CD10, PDGFR a, CD49b and CD49d at very high level while CD106 is expressed at very low level. Moreover, STRO-1 and CD34 are not detected (Table 1). FIG. 12 represents a bar chart of a comparison of stem/progenitor cells (EPCs) according to the invention with fibroblasts after 21 days of osteogenic stimulation (a) and an agarose gel showing gene expression of EPCs after osteogenic induction (b). To investigate the osteogenic potential of EPCs, cells were cultured in medium for osteogenic induction (dexamethasone, ascorbic acid-2 phosphate and β-glycerol phosphate). Quantitative Ca²⁺ analysis showed a high Ca²⁺ accumulation compared to fibroblasts (a). Gene expression analysis demonstrated that alkaline phosphatase (ALP) was expressed all time whereas osteocalcin (OCN) turns out to be upregulated under differentiation conditions (b).

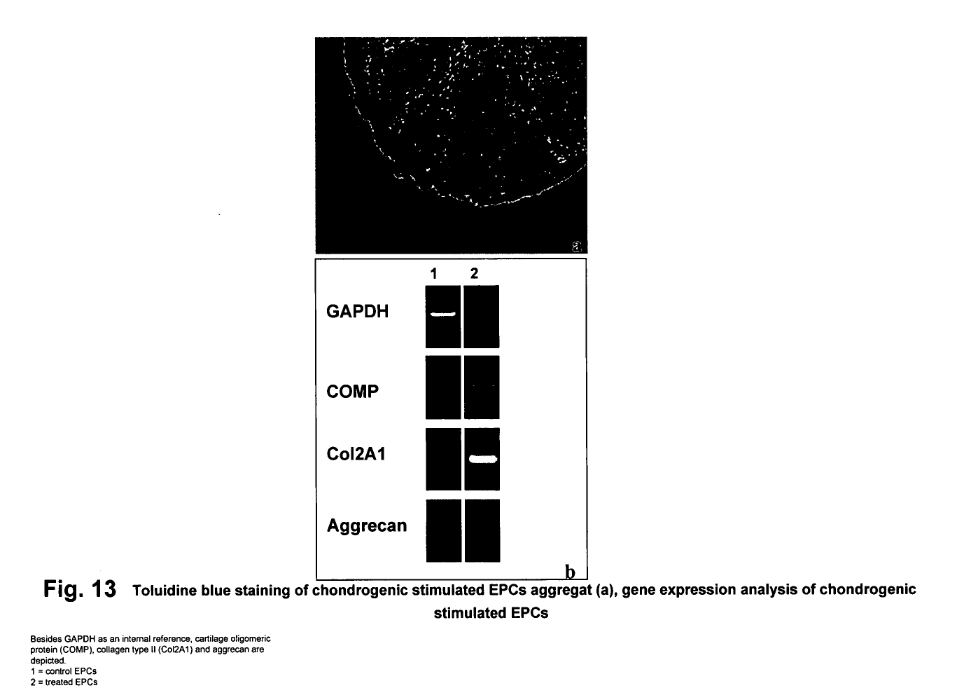

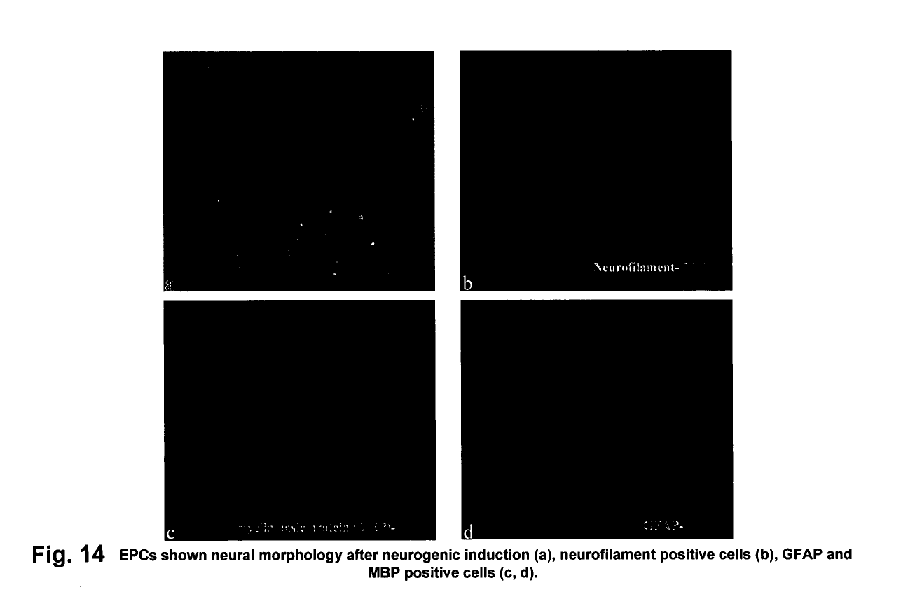

FIG. 13 represents a micrograph of toluidine blue staining of chondrogenic stimulated EPCs aggregate (a) and a gene expression analysis of chondrogenically stimulated EPCs, expression of GAPDH (internal reference), cartilage oligomeric protein (COMP), collagen type 11 (Col2A1) and aggrecan (b). EPCs were assayed for their chondrogenic potential using a well established aggregate culture system. When cultured under micromass condition, cells formed nodules with a well organized ECM-sulphated proteoglycans are presented. The cells were reorganized into a cartilage-like tissue and stained positive with toluidine blue (a). RT-PCR analysis reveals upregulation of chondrogenic specific markers as type 11 collagen, aggrecan and COMP (cartilage oligomatrix protein). These data reflected a chondrogenic fate of cultures when treated with differentiation medium (b). FIG. 14 represents micrographs of stem/progenitor cells (EPCs) according to the invention after neurogenic induction (a), neurofilament positive cells (b), GFAP and MBP positive cells (c, d). EPCs were expanded to high confluency and cultivated in serum free medium with B27, bFGF and EGF. After 4-7 days, free floating spheres were seeded on laminin/PDL coated cover slides. Under neurogenic induction cells showed a neuro-like morphology (a). The neurofilament-positive subpopulation of the EPCs can be detected by further incubation in neurogenic medium (b). Astrocytes marker GFAP and oligodendrocytes marker myelin basic protein (MBP) were detected in neurogenic induction medium cultured EPCs (c,d).

### LIST OF REFERENCE NUMBERS

1 Tooth
2 Dental follicle
3 Pad-like tissue
4 Crown
5 Root
6 Tooth
7 Operculum
8 Dental follicle
9 Pad-like tissue
10 Dental papilla
11 Alveolar bone
12 Dental papilla
13 Dental follicle
14 Crown
15 Operculum
16 Pad-like tissue
17 Root

1. Method for isolating non-embryonic stem cells comprising
identifying a pad-like tissue located underneath the dental papilla in immediate vicinity of the apical side of a surgically removed immature tooth or wisdom tooth which is in a development stage between the appearance of the bony alveolar fundus and the end of the formation of the root of said tooth or wisdom tooth,
separating said pad-like tissue from said dental papilla and/or said root, and
singularising cells by disintegrating said pad-like tissue.

2. Method according to claim 1, wherein said pad-like tissue is separated from said dental papilla and/or said root by dissection, in particular by cutting along the macroscopically visible border between the dental papilla and the pad-like tissue.

3. Method according to claim 1, wherein said pad-like tissue is disintegrated by enzymatic digestion.

4. Method according to claim 1, wherein said tooth or wisdom tooth is a mammal tooth.

5. Method according to claim 1, wherein said singularised cells are transfected with a nucleic acid.

6. Method according to claim 1, wherein said singularised cells are stimulated to differentiate into bone-forming cells or craniofacial cells or tissue.

7. Method according to claim 1, wherein said singularised cells are stimulated to differentiate into neurons or nerve tissue.

8. Non-embryonic stem cell isolated from a pad-like tissue located underneath the dental papilla in immediate vicinity of the apical side of an immature tooth or wisdom tooth which is in a stage of development between the appearance of the bony alveolar fundus and the end of the formation of the root of said tooth or wisdom tooth.

9. Non-embryonic stem cell according to claim 8, wherein said stem cell is a mammalian cell.

10. Non-embryonic stem cell, which is isolated by the method according to claim 1.

11. Non-embryonic stem cell according to claim 8, which is transfected with a nucleic acid, in particular DNA, preferably comprising at least one gene of interest and/or at least one marker sequence.

12. Cell culture or cell structure comprising at least one non-embryonic stem cell according to claim 8.

13. Pharmaceutical composition comprising at least one non-embryonic stem cell according to claim 8.

14. (canceled)

15. Method according to claim 2, wherein said pad-like tissue is disintegrated by enzymatic digestion.

16. Method according to claim 4, wherein said tooth or wisdom tooth is a human tooth.

17. Method according to claim 5, wherein said nucleic acid is DNA.

18. Method according to claim 5, wherein said nucleic acid comprises at least one gene of interest and/or at least one marker sequence.

19. Non-embryonic stem cell according to claim 8, wherein said stem cell is a human cell.

20. Method for repairing or replacing damaged or destroyed tissue in vivo, comprising
providing stem cells isolated by the method of claim 1, and
repairing or replacing damaged or destroyed tissue in vivo.

21. Method according to claim 21, wherein said damaged or destroyed tissue is ectomesenchymal tissue.
